(19) United States
(12) Patent Application Publication
(10) Pub. No.: US 2006/0005642 A1
(43) Pub. Date: Jan. 12, 2006

(54) Rapid aging of fiber glass insulation to determine product fitness

(76) Inventor: Ward Thomas Hobert, Golden, CO (US)

Correspondence Address: Johns Manville, 10100 West Ute Avenue, Littleton, CO, 80127, US

(21) Appl. No.: 10/888,824

(22) Filed: Jul. 9, 2004

(51) Int. Cl. G01N 33/00

(52) U.S. Cl. 73/866

(57) ABSTRACT

A system for evaluating the long term performance of glass fiber insulation is disclosed. Samples of insulation are conditioned for a period of up to about 30 days at conditions of heat up to about 50° C. and humidity up to 100% Relative Humidity and performance tested.

### TECHNICAL FIELD

The method relates to a method for rapidly aging fiberglass products to determine their fitness for use. In one embodiment, fiberglass insulation is stored at an elevated temperature and humidity and then subjected to one or more tests to determine its suitability for use as insulation.

### BACKGROUND OF THE INVENTION

Fibrous glass products such as fiberglass insulation generally comprise matted glass fibers bonded together by a binder that is often a cured thermoset polymeric material. Molten streams of glass are drawn into fibers of random lengths and blown into a forming chamber where they are randomly deposited as a mat onto a traveling conveyor. The fibers, while in transit in the forming chamber, and while still hot from the drawing operation, are sprayed with the binder(often aqueous-based). The coated fibrous mat is transferred to a curing oven where heated air, for example, is blown through the mat to cure the binder and rigidly bond the glass fibers together.

Fiberglass binders have a variety of uses ranging from stiffening applications where the binder is applied to woven or non-woven fiberglass sheet goods and cured, producing a stiffer product; thermo-forming applications wherein the binder resin is applied to sheet or lofty fibrous product following which it is dried and optionally B-staged to form an intermediate but yet curable product; and to fully cured systems such as building insulation.

Fiberglass binders used in the present sense should not be confused with matrix resins which are an entirely different and non-analogous field of art. While sometimes termed “binders”, matrix resins act to fill the entire interstitial space between fibers, resulting in a dense, fiber reinforced product where the matrix must translate the fiber strength properties to the composite, whereas “binder resins” as used herein are not space-filling, but rather coat only the fibers, and particularly the junctions of fibers. Fiberglass binders also cannot be equated with paper or wood product “binders” where the adhesive properties are tailored to the chemical nature of the cellulosic substrates. Many such resins, e.g. urea/formaldehyde and resorcinol/formaldehyde resins, are not suitable for use as fiberglass insulation binders. One skilled in the art of fiberglass binders would not look to cellulosic binders to solve any of the known problems associated with fiberglass binders.

Binders useful in fiberglass insulation products generally require a low viscosity in the uncured state, yet characteristics so as to form a rigid thermoset polymeric mat for the glass fibers when cured. A low binder viscosity in the uncured state is required to allow the mat to be sized correctly. Also, viscous binders tend to be tacky or sticky and hence they lead to accumulation of fiber on the forming chamber walls. This accumulated fiber may later fall onto the mat causing dense areas and product problems. A binder which forms a rigid matrix when cured is required so that a finished fiberglass thermal insulation product, when compressed for packaging and shipping, will recover to its specified vertical dimension when installed in a building.

From among the many thermosetting polymers, numerous candidates for suitable thermosetting fiber-glass binder resins exist. But, binder-coated fiberglass products are often of the commodity type, and thus cost becomes a driving factor, generally ruling out such resins as thermosetting polyurethanes, epoxies, and others. Due to their excellent cost/performance ratio, the resins of choice in the past have been phenol/formaldehyde resins. Phenol/formaldehyde resins can be economically produced, and can be extended with urea prior to use as a binder in many applications. Such urea-extended phenol/formaldehyde binders have been the mainstay of the fiberglass insulation industry for years.

Over the past several decades, however, minimization of volatile organic compound emissions (VOCs) both on the part of the industry desiring to provide a cleaner environment, as well as by Federal regulation, hassled to extensive investigations into not only reducing emissions from the current formaldehyde-based binders, but also into candidate replacement binders. For example, subtle changes in the ratios of phenol to formaldehyde in the preparation of the basic phenol/formaldehyde resole resins, changes in catalysts, and addition of different and multiple formaldehyde scavengers, has resulted in considerable improvement in emissions from phenol/formaldehyde binders as compared with the binders previously used. However, with increasing stringent Federal regulations, more and more attention has been paid to alternative binder systems which are free from formaldehyde.

One particularly useful formaldehyde-free binder system employs a binder comprising a polycarboxy polymer and a polyol. Formaldehyde-free resins are those which are not made with formaldehyde or formaldehyde-generating compounds. Formaldehyde-free resins do not emit appreciable levels of formaldehyde during the insulation manufacturing process and do not emit formaldehyde under normal service conditions. Use of this binder system in conjunction with a catalyst, such as an alkaline metal salt of a phosphorous-containing organic acid, results in glass fiber products that exhibit excellent recovery and rigidity properties.

Glass fiber based insulation (“insulation”) is manufactured year-round and shipped to locations through out the United States and the world. Accordingly, insulation is subject to various conditions of temperature and humidity from manufacturer to shipping to storage and subsequent installation. Binders may be affected by transportation and storage under conditions of high temperatures and humidity in unpredictable ways when used in glass fiber insulation. The performance of batts of insulation are subject to packaging effects such as settling, and compression, and these are also in turn effected by humidity, temperature and the like. Because of the unknown properties of glass fiber insulation compositions, the present invention is directed to novel, rapid methods of aging and testing for the effects of heat and humidity on performance properties of glass fiber based insulation.

### BRIEF SUMMARY OF THE INVENTION

The present invention is directed to a system and method which provides rapid aging of glass fiber based products followed by qualitative and/or qualitative testing of the products. The properties of the aged product are then evaluated using one or more test methods.

The invention provides a method for estimating the properties of rapidly aged glass fiber insulation, comprising the steps of: conditioning a sample of glass fiber based insulation under conditions of constant temperature and humidity for a specific time period.

The humidity used to age the product can range from about 80 to about 100%, with from 85 to 95% preferred with a relative humidity (RH) of about 90% preferred. The temperature used in the method should be from about 25° C. to 50° C. with from 25° C. to about 35° C. preferred and about 32° C. most preferred. The product should be stored at an elevated temperature and humidity for a sufficient period to give rise to mechanisms that might adversely affect the properties of the product. This is typically a period of from about 2 to about 30 days with from about 7 to about 28 days preferred.

Following exposure of the fiberglass to heat and humidity, the fiberglass product is then tested to determine its suitability using one or more well known testing procedures. Among the tests that can be used to determine suitability are tests for rigidity and thickness recovery.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated that the conception and specific embodiment disclosed may be readily utilized as a basis for modifying or designing other structures for carrying out the same purposes of the present invention. It should also be realized that such equivalent constructions do not depart from the invention as set forth in the appended claims. The novel features which are believed to be characteristic of the invention, both as to its organization and method of operation, together with further objects and advantages will be better understood from the following description.

### DETAILED DESCRIPTION OF THE INVENTION

The invention is a method for rapidly aging fiberglass products such as insulation batts to determine there fitness for use. The methods can be used to age fiberglass products prepared with traditional phenol-formaldehyde-based binder composition as well as those prepared with novel formaldehyde-free binder compositions. As used herein, the term “formaldehyde-free” means that the resin or binder composition is substantially free of formaldehyde and/or does not liberate formaldehyde as a result of drying or curing.

The method involves subjecting a sample of a binder-coated fiberglass product to heat and humidity for a specified period of time to accelerate the aging process. After the sample has been subjected to rapid aging, the sample is then tested using one or more tests to determine the product's fitness for use. The inventive method provides fiberglass product manufacturers an effective means for predicting the ability of their products to withstand the environmental conditions the products will see during transport and storage as well as during actual use.

The fiberglass products should be exposed to temperature and humidity high enough to accelerate aging effects, but not so high as to give rise to mechanisms of degradation that do not exist in actual storage and usage conditions. It has been unexpectedly found that conditions of about 30° to about 50° C., preferably about 30° C. to 35° C. and a humidity range of about 85% RH to about 95% RH, preferably about 90% relative humidity are useful to enhance aging of glass fiber based insulation. Conditioning for periods from as short as about 2 days have been found useful in discerning accelerated aging. In preferred embodiments the testing is done after exposure periods of from about 2 days to about 30 days of accelerated aging with about 7 days to about 28 days is most preferred.

As noted above, any binder-coated fiberglass product can be tested using this method. The test is particularly useful in evaluating binder-coated fiberglass insulation. When testing insulation, the sample tested is packaged and unpackaged. When the product is tested packaged, it is preferably to open or perforate the packaging to allow more rapid diffusing of the moisture-laden air into the product. This is true for any packaged fiberglass product tested using this method.

Following the rapid aging step, the product is then tested to determine its fitness for use. This is done using one or more fitness tests known to those skilled in the art. For example, in the case of fiberglass insulation batts, performance test include, but are not limited to, tests for thickness recovery. This test can be done alone or in combination to determine if the fiberglass insulation will meet the customer's expectation.

One thickness recovery test that can be used is that defined in ASTM method 167C Standard Test Methods for Thickness and Density of Blanket or Batt thermal Insulation. Modifications of this procedure can also be used.

While the rigidity test is preferred, other performance relative tests known in the art can be used in the practice of this invention.

### EXAMPLE 1

Three commercial samples of R19 fiberglass insulation were tested using a modified version of ASTMC 167. For each sample, the initial measurement was made by taking a 48 inch (122 cm) long specimen of the product, and dropping the specimen onto a flat surface from a height of 18 inches (45.7 cm) two times on each long edge for a total of four drops per specimen. The thickness of the specimen is then measured using a pin and disk. In this method, a pin is used to penetrate the specimen perpendicular to the flat surface. When the pin reaches the flat surface, the disk is allowed to slide down the pin to the surface of the specimen under its own weight. Holding the disk in place, the pin and disk are removed and the distance from the disk to the point of the pin was measured. Four measurements were taken across the surface of the specimen and the measurements are then averaged. This was repeated and the averages for each specimen combined and averaged. The specimens yielded average recovery measurements of 4.9, 5.1 and 4.9 respectively.

Samples of the same products were then stored at 90% RH and 33° C. for seven days. The samples were again tested using the procedure outlined above. The results after aging were 4.59 (11.7 cm), 4.59 (11.7 cm), and 4.63 (11.8 cm) inches respectively.

Although the present invention and its advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

1. A method for evaluating fiberglass products comprising:
exposing a sample of a fiberglass product at an elevated temperature and humidity;
evaluating the performance of the fiberglass product following the exposure to elevated beat and humidity.

2. (canceled)

3. (canceled)

4. The method of claim 1 wherein the temperature is about 33° C. and the humidity is about 90%.

5. (canceled)

6. The method of claim 1 where the evaluation of the sample is conducted using a thickness recovery test.

7. The method of claim 1 wherein the fiberglass product is fiberglass insulation.

8. The method of claim 7 wherein the fiberglass insulation comprises a formaldehyde-free binder.

9. (canceled)

10. (canceled)

11. (canceled)

12. (canceled)

13. (canceled)

14. (canceled)
